(19) United States
(12) Patent Application Publication
(10) Pub. No.: US 2019/0096524 A1
(43) Pub. Date: Mar. 28, 2019

(54) MECHANISM OF ACTION DERIVATION FOR DRUG CANDIDATE ADVERSE DRUG REACTION PREDICTIONS

(71) Applicant: INTERNATIONAL BUSINESS MACHINES CORPORATION, Armonk, NY (US)

(72) Inventors: Jianying HU, Yorktown Heights, NY (US); Heng LUO, Yorktown Heights, NY (US); Janu VERMA, Yorktown Heights, NY (US); Ping ZHANG, Yorktown Heights, NY (US)

(21) Appl. No.: 15/801,714

(22) Filed: Nov. 2, 2017

Related U.S. Application Data
(63) Continuation of application No. 15/715,548, filed on Sep. 26, 2017.

(51) Int. Cl. G06F 19/00; G06N 99/00

(52) U.S. Cl. CPC G16H 50/20; G06N 99/005; G06F 19/704

(57) ABSTRACT

Embodiments include methods, systems, and computer program products for generating a mechanism of action hypothesis. Aspects include receiving a drug candidate data along with a plurality of predicted adverse drug reactions (ADRs) associated with the drug candidate data. Aspects include receiving a drug pathway data for the drug candidate and adverse drug reaction pathway data for each of the plurality of predicted adverse drug reactions. Aspects include building a pathway network, wherein the pathway network includes a plurality of drug pathway nodes, a plurality of ADR pathway nodes, and a plurality of pathway connections. Aspects also include generating a pathway output.

### DOMESTIC AND/OR FOREIGN PRIORITY

This application is a continuation of U.S. application Ser. No. 15/715,548, titled “Mechanism of Action Derivation for Drug Candidate Adverse Drug Reaction Predictions” filed Sep. 26, 2017, the contents of which are incorporated by reference herein in its entirety.

### BACKGROUND

The present invention relates in general to adverse drug reaction prediction, and more specifically, to mechanism of action hypothesis derivation for drug candidate-adverse drug reaction predictions.

Adverse drug reactions (ADRs) are unintended and potentially harmful reactions caused by normal uses of drugs. ADRs represent a significant public health problem all over the world. Predicting ADRs can be extremely valuable and important for safe drug development and precision medicine. Machine learning models have been developed to aid in the development and assessment of drug candidates, for instance to aid in the prediction of adverse drug reactions ADRs. Though some machine learning models can effectively predict ADRs, such models can lack biological interpretation and the outputs can be difficult to explain from a biological perspective. For instance, an output of a machine learning model can consist of an identification of a potential ADR and a numerical value representing a statistical likelihood associated with the ADR.

### SUMMARY

In accordance with embodiments of the invention, a computer-implemented method for generating a mechanism of action hypothesis for an adverse drug reaction is provided. A non-limiting example of the method includes receiving, by a processor, drug candidate data that identifies a drug candidate, along with a plurality of predicted adverse drug reactions associated with the drug candidate data. The method also includes receiving, by the processor, drug pathway data for the drug candidate. The method also includes receiving, by the processor, adverse drug reaction pathway data for each of the plurality of predicted adverse drug reactions. The method also includes building, by the processor, a pathway network, wherein the pathway network includes a plurality of drug pathway nodes, a plurality of adverse drug reaction pathway nodes, and a plurality of pathway connections. The method also includes generating a pathway output.

In accordance with embodiments of the invention, a computer program product for generating a mechanism of action hypothesis for an adverse drug reaction is provided. A non-limiting example of the computer program product includes a computer readable storage medium readable by a processing circuit. The computer readable storage medium stores program instructions for execution by the processing circuit for performing a method. The method includes receiving drug candidate data that identifies a drug candidate, along with a plurality of predicted adverse drug reactions associated with the drug candidate. The method also includes receiving a drug pathway data for the drug candidate. The method also includes receiving adverse drug reaction pathway data for each of the plurality of predicted adverse drug reactions. The method also includes building a pathway network, wherein the pathway network includes a plurality of drug pathway nodes, a plurality of adverse drug reaction pathway nodes, and a plurality of pathway connections. The method also includes generating a pathway output.

In accordance embodiments of the invention, a processing system for generating a mechanism of action hypothesis for an adverse drug reaction includes a processor in communication with one or more types of memory. The processor is configured to receive drug candidate data that identifies a drug candidate, along with a plurality of predicted adverse drug reactions associated with the drug candidate data. The processor is also configured to receive a drug pathway data for the drug candidate. The processor is also configured to receive adverse drug reaction pathway data for each of the plurality of predicted adverse drug reactions. The processor is also configured to build a pathway network, wherein the pathway network includes a plurality of drug pathway nodes, a plurality of adverse drug reaction pathway nodes, and a plurality of pathway connections. The processor is also configured to generate a pathway output.

In accordance embodiments of the invention, a computer-implemented method for displaying a mechanism of action hypothesis for an adverse drug reaction is provided. A non-limiting example of the method includes building a pathway network between drug candidates and adverse drug reactions (ADRs), wherein the pathway network includes a plurality of drug pathway nodes for a drug, a plurality of ADR nodes for an associated ADR, and connections between the drug pathways and ADR pathways. The method also includes displaying the plurality of drug pathway nodes in a drug pathway region on a graphical user interface. The method also includes displaying a plurality of ADR pathway nodes in an ADR pathway region on the graphical user interface. The method also includes displaying a plurality of pathway connections by connecting one or more of the drug pathway nodes to one or more of the ADR pathway nodes by one or more lines, wherein the relative thickness of each of the lines reflects the statistical significance of a drug pathway-ADR pathway connection.

In accordance with embodiments of the invention, a system for generating a mechanism of action hypothesis is provided. A non-limiting example of the system includes an input including a drug structure input, a drug pathway input, and an adverse drug reaction (ADR) input. The system also includes a pathway analysis engine including an ADR prediction module, a pathway harvest module, a pathway network formation module, and a network connection ranking module. The system also includes a system output interface.

### BRIEF DESCRIPTION OF THE DRAWINGS

The subject matter of the present invention is particularly pointed out and distinctly claimed in the claims at the conclusion of the specification. The foregoing and other features and advantages of the one or more embodiments described herein are apparent from the following detailed description taken in conjunction with the accompanying drawings in which:

FIG. 1 depicts a cloud computing environment according to embodiments of the present invention.

FIG. 2 depicts abstraction model layers according to embodiments of the present invention.

FIG. 3 depicts a computer system according to embodiments of the present invention.

FIG. 4 depicts an exemplary machine learning model interface for drug-adverse drug reaction (ADR) prediction for use in embodiments of the invention.

FIG. 5 depicts an exemplary system according to embodiments of the present invention.

FIG. 6 depicts an exemplary system interface according to embodiments of the present invention.

FIG. 7 depicts a flow diagram illustrating an exemplary method according to embodiments of the present invention.

FIG. 8 depicts a flow diagram illustrating an exemplary method according to embodiments of the present invention.

FIG. 9 depicts aspects of an exemplary system according to embodiments of the present invention.

FIG. 10 depicts aspects of an exemplary system according to embodiments of the present invention.

FIG. 11 depicts aspects of an exemplary system according to embodiments of the present invention.

FIG. 12 depicts aspects of an exemplary system according to embodiments of the present invention.

FIG. 13 depicts aspects of an exemplary system according to embodiments of the present invention.

FIG. 14 depicts aspects of an exemplary system according to embodiments of the present invention.

FIG. 15 depicts aspects of an exemplary system according to embodiments of the present invention.

FIG. 16 depicts aspects of an exemplary system according to embodiments of the present invention.

FIG. 17 depicts aspects of an exemplary system according to embodiments of the present invention.

FIG. 18 depicts aspects of an exemplary system according to embodiments of the present invention.

### DETAILED DESCRIPTION

It is understood in advance that although this description includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model can include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but can be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It can be managed by the organization or a third party and can exist on-premises or off-premises.

Community cloud: the cloud infrastructure is shared by several organizations and supports a specific community that has shared concerns (e.g., mission, security requirements, policy, and compliance considerations). It can be managed by the organizations or a third party and can exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure including a network of interconnected nodes.

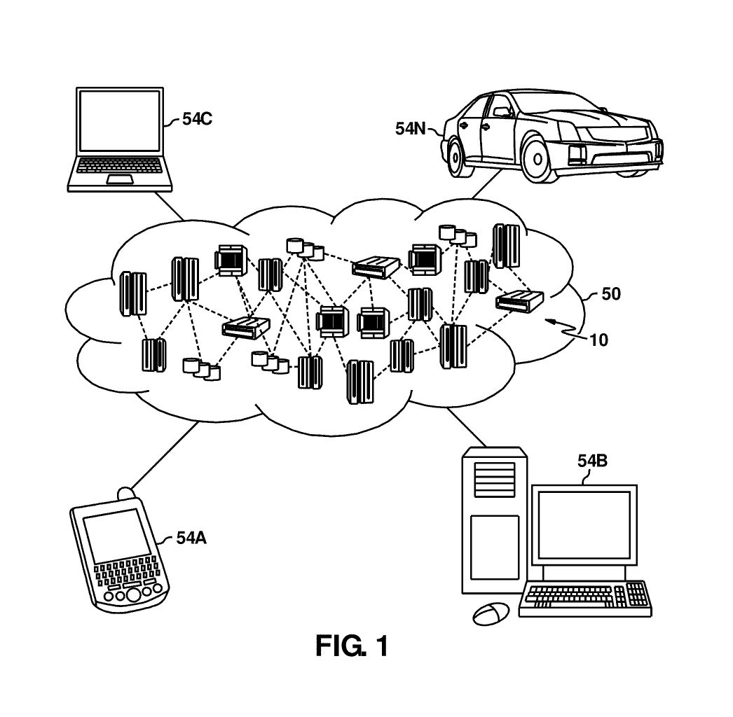

Referring now to FIG. 1, illustrative cloud computing environment 50 according to one or more embodiments of the present invention is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N can communicate. Nodes 10 can communicate with one another. They can be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

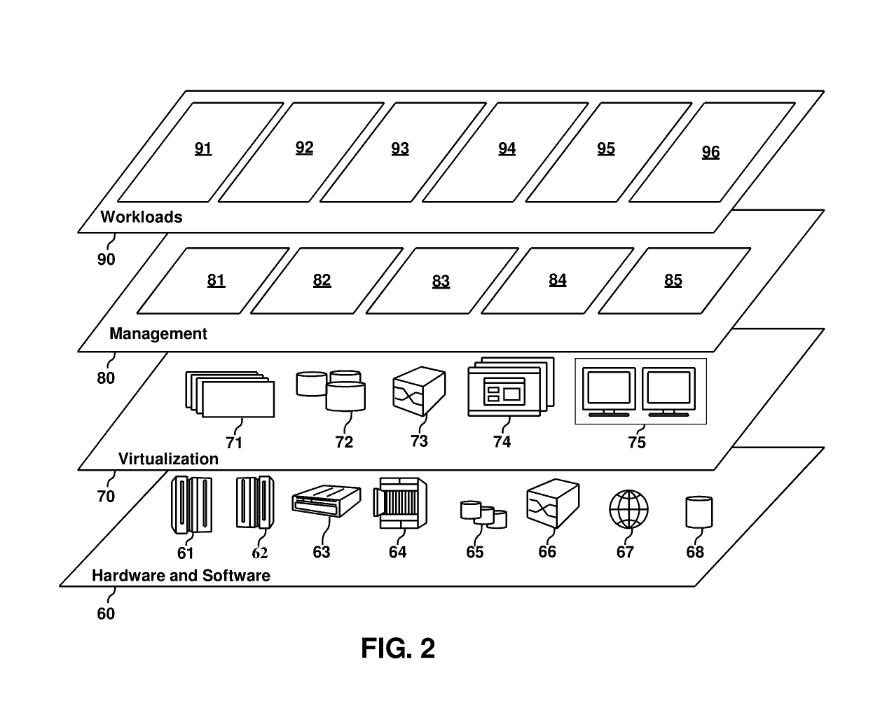

Referring now to FIG. 2, a set of functional abstraction layers provided by cloud computing environment 50 (FIG. 1) according to one or more embodiments of the present invention is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments of the invention, software components include network application server software 67 and database software 68.

Virtualization layer 70 provides an abstraction layer from which the following examples of virtual entities can be provided: virtual servers 71; virtual storage 72; virtual networks 73, including virtual private networks; virtual applications and operating systems 74; and virtual clients 75.

In one example, management layer 80 can provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources can include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment can be utilized. Examples of workloads and functions which can be provided from this layer include: mapping and navigation 91; software development and lifecycle management 92; virtual classroom education delivery 93; data analytics processing 94; transaction processing 95; and adverse drug reaction analysis 96.

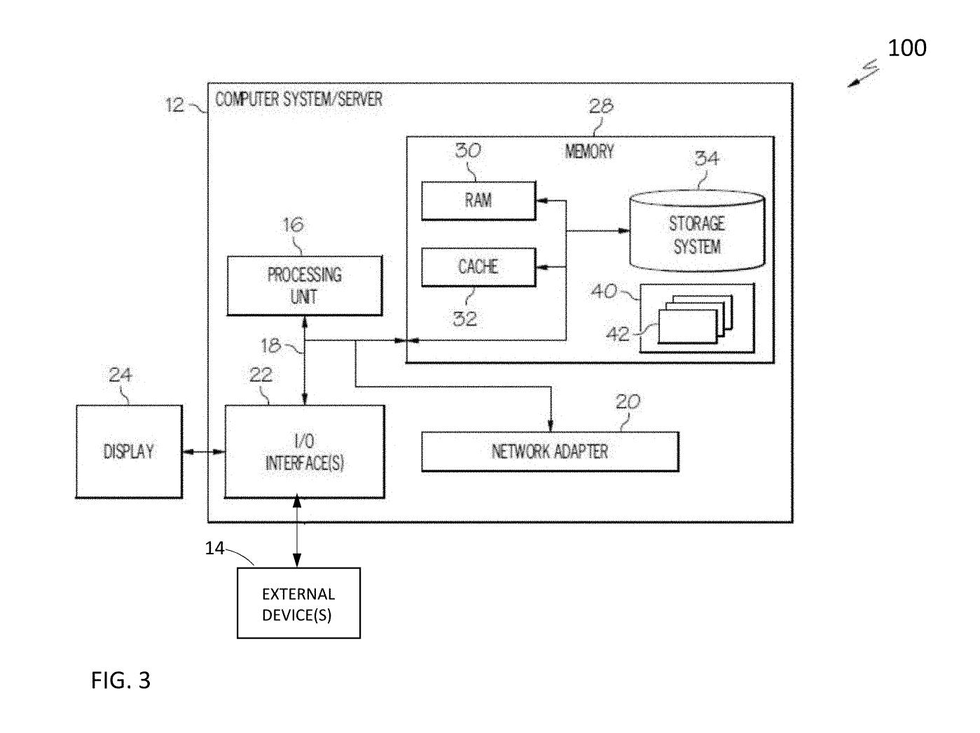

Referring now to FIG. 3, a schematic of a cloud computing node 100 included in a distributed cloud environment or cloud service network is shown according to one or more embodiments of the present invention. The cloud computing node 100 is only one example of a suitable cloud computing node and is not intended to suggest any limitation as to the scope of use or functionality of embodiments of the invention described herein. Regardless, cloud computing node 100 is capable of being implemented and/or performing any of the functionality set forth hereinabove.

In cloud computing node 100 there is a computer system/server 12, which is operational with numerous other general purpose or special purpose computing system environments or configurations. Examples of well-known computing systems, environments, and/or configurations that can be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 can be described in the general context of computer system-executable instructions, such as program modules, being executed by a computer system. Generally, program modules can include routines, programs, objects, components, logic, data structures, and so on that perform particular tasks or implement particular abstract data types. Computer system/server 12 can be practiced in distributed cloud computing environments where tasks are performed by remote processing devices that are linked through a communications network. In a distributed cloud computing environment, program modules can be located in both local and remote computer system storage media including memory storage devices.

As shown in FIG. 3, computer system/server 12 in cloud computing node 100 is shown in the form of a general-purpose computing device. The components of computer system/server 12 can include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media can be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 can further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 can include at least one program product having a set (e.g., at least one) of program modules that are configured to carry out the functions of embodiments of the invention.

Program/utility 40, having a set (at least one) of program modules 42, can be stored in memory 28 by way of example, and not limitation, as well as an operating system, one or more application programs, other program modules, and program data. Each of the operating system, one or more application programs, other program modules, and program data or some combination thereof, can include an implementation of a networking environment. Program modules 42 generally carry out one or more functions and/or methodologies in accordance with some embodiments of the present invention.

Computer system/server 12 can also communicate with one or more external devices 14 such as a keyboard, a pointing device, a display 24, etc., one or more devices that enable a user to interact with computer system/server 12, and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

Turning now to an overview of technologies that are more specifically relevant to aspects of the invention, adverse drug reactions (ADRs) are potentially adverse clinical conditions that can result from taking medications at normal doses. Drug development companies and researchers continuously seek to identify ADRs and potential ADRs early in drug development to reduce the time, cost, and patient risk associated with development activities.

To identify and predict ADRs, computational methods are available, such as machine learning models on structural descriptors, similarity analysis of molecular docking profiles, network-based approaches, and data mining of electronic health records. Although some methods can generate ADR predictions, information concerning underlying biological methods are not generated. Such models, for instance, can output a score quantifying the association of a drug or drug candidate with an ADR. As used herein, “drug” and “drug candidate” are interchangeable and are intended to include any chemical under investigation as a

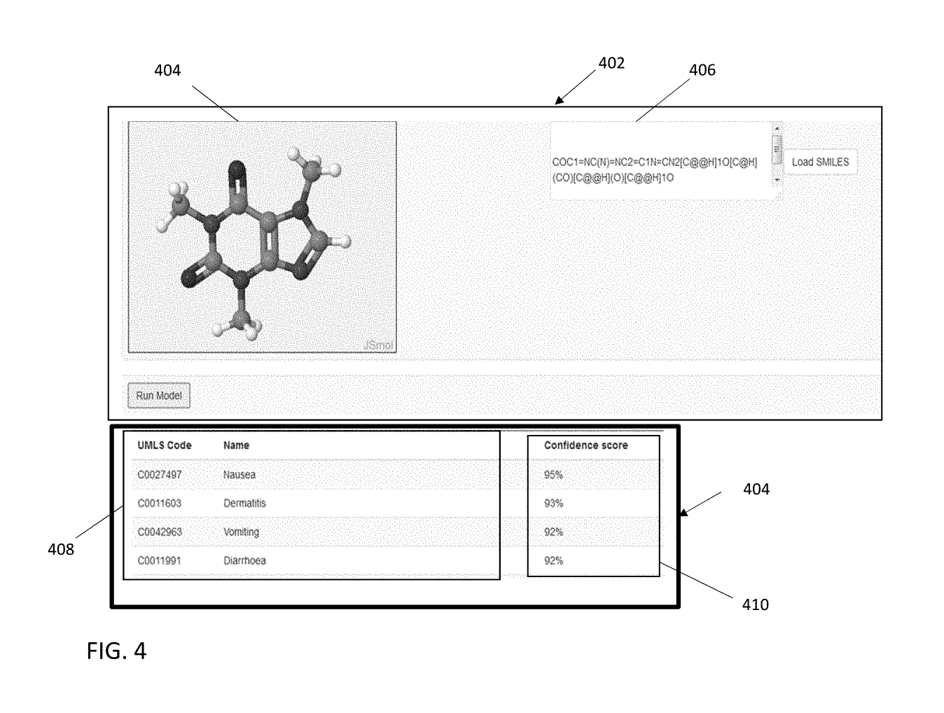

FIG. 4 depicts an exemplary drug-ADR prediction interface of a machine learning model for use in embodiments of the invention. In the example shown, an input interface 402 can include a three dimensional chemical structure depiction 404 and/or a chemical descriptor input 406, such as a simplified molecular-input line-entry system (SMILES) input. An output 404 displays the results of the machine learning model drug-ADR prediction, which can include an ADR listing 408, which depicts the resultant predicted ADRs based upon the chemical descriptor, and a confidence score listing 410 associated with the drug-ADR predictions. What remains unclear is the biological interpretation of such models, including a rationale or reason why a particular ADR has a higher association score for one drug candidate than for another. Deeper understanding and interpretation of drug-ADR associations are highly desirable.

Turning now to an overview of the aspects of the invention, one or more embodiments of the invention address the above-described shortcomings of the prior art by providing a comparison of metabolic pathways of ADRs and drugs or drug candidates, thereby providing a hypothesis of mechanism of action for drug-ADR associations. One more embodiments of the invention can provide a visual representation of statistical associations between drugs and ADRs, providing enhanced output for a deeper understanding and interpretation of drug-ADR associations. As used herein, pathway data and metabolic pathway data are used interchangeably and include genes, gene interaction(s), gene enrichment, and the like, associated or linked with a drug (e.g., drug pathway data) and/or an ADR (e.g., ADR pathway data), as indicated. In some embodiments of the invention, the visual representation and/or metabolic pathway comparison provides a hypothesis of the mechanism of action for an ADR associated with a drug. By showing shared pathways, and common genes in those pathways along with relative significance, drug developers can readily determine the likely cause of a predicted ADR and thereby streamline their research efforts and develop safe and efficacious drugs more quickly.

The above-described aspects of the invention address the shortcomings of the prior art by harvesting pathway information from gene expression analysis and from the literature for the drugs and ADRs of drug-ADR pairs generated with prediction models and determining and quantifying pathway connections between the drug-ADR pairs. Pathway connections between drugs and ADRs can be statistically analyzed and ranked, for example by Jaccard index. Top connections can serve as candidate mechanism of action hypothesis of how a drug or drug candidate induces ADRs. Some embodiments of the invention provide a visual diagram, such as a Sankey diagram, Venn diagram, UpSet visualization etc. that visually captures and provides the flow of information from drugs to drug pathways to ADR pathways to ADRs. Such visual diagrams can provide explicit displays of the rankings of various comparisons using, for example, thickness of edges (i.e., connectors between a drug and an ADR), making the results of the metabolic comparison easy to observe and compare, providing superior drug candidate evaluation. In some embodiments of the invention, an interactive user interface is provided, in which a user can interact with displayed edges or connectors to obtain a listing of common genes between a drug and an ADR along with statistical similarity, such as Jaccard similarity.

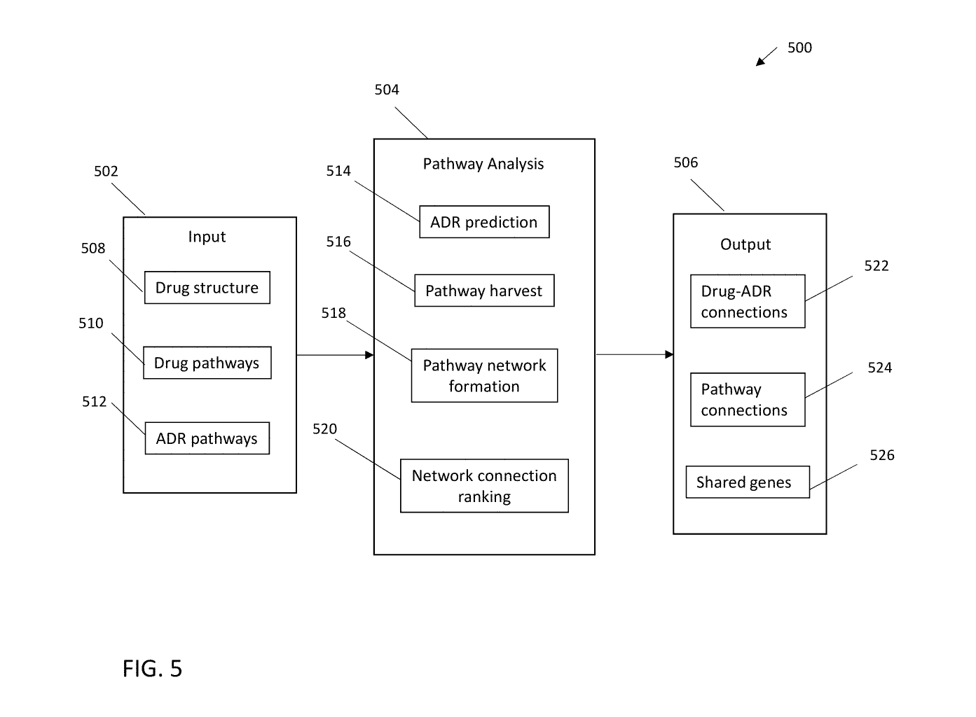

Turning now to a more detailed description of aspects of the present invention, FIG. 5 depicts a schematic of an exemplary system 500 according to one or more embodiments of the invention. The system 500 can include an input 502, a pathway analysis engine 504, and an output 506, configured and arranged as shown. The input 502 can include a drug structure input 508, drug pathways input 510, and ADR pathways input 512.

The drug structure input 508 can be adapted to receive two dimensional or three dimensional chemical structure information in any suitable feature vector or format based upon its chemical structure or substructures, such as SMILES, the IUPAC International Chemical Identifier (InChI™) key, and the like. The drug structure input 508, in some embodiments of the invention, can receive drug features, such as structure descriptors (e.g., from PubChem), labels for instance from the SIDER database, and related information.

Drug pathways input 510 can be adapted to receive pathway information for a drug from gene expression analysis and the literature. In some embodiments of the invention, drug pathways input 510 is adapted to receive pathway data from gene expression analysis of drug effect. Any database providing drug pathway data can be accessed in accordance with embodiments of the invention, such as Drug-Path and/or the small molecule pathway database (SMPDB).

ADR pathways input 512 can be adapted to receive pathway information for an ADR curated from the literature. ADR pathways input can include structured and unstructured data and can include, but is not limited to, data published in journal articles in a narrative format. Any database providing drug pathway data can be accessed in accordance with embodiments of the invention, including for example the Kyoto Encyclopedia of Genes and Genomes (KEGG), Reactome Pathway Database, and/or the Protein Analysis through Evolutionary Relationships (PANTHER) system.

The pathway analysis engine 504 can include, for example, an ADR prediction module 514, a pathway harvest module 516, a pathway network formation module 518, and a network connection ranking module 520. An ADR prediction module 514 can predict one or more ADRs for a given drug, for instance by using a machine learning model or any other predictive model useful for generating predicted ADRs for a drug. The pathway harvest module 516 can harvest pathways for a drug from the drug pathways input 510 and from the ADR pathways input 512. The pathway network formation module 518 can identify and statistically evaluate connections between drug pathways and ADR pathways, for example using a Jaccard Index, IDF-normalized cosine, intersection, and the like. In some embodiments of the invention, a statistical method that is not impacted by the number of genes in the pathway, such as a Jaccard Index, is used. The network connection ranking module 516 can rank the connections between drug pathways and ADR pathways. In some embodiments of the invention, the network connection ranking module 516 can generate a visualized output for the connections and/or flow of information from drugs to drug pathways to ADR pathways to ADRs, such as a Sankey diagram, providing superior drug candidate evaluation.

The output 506 can include, for example, drug-ADR connections 522, such as drug-ADR pairs associated by a machine learning model, pathway connections 524, and shared genes 526 between each drug and its associated ADRs having common or shared pathways. One or more of the drug-ADR connections 522, pathway connections 524, and shared genes con include interactive and/or dynamic components to enhance the evaluation of a drug candidate.

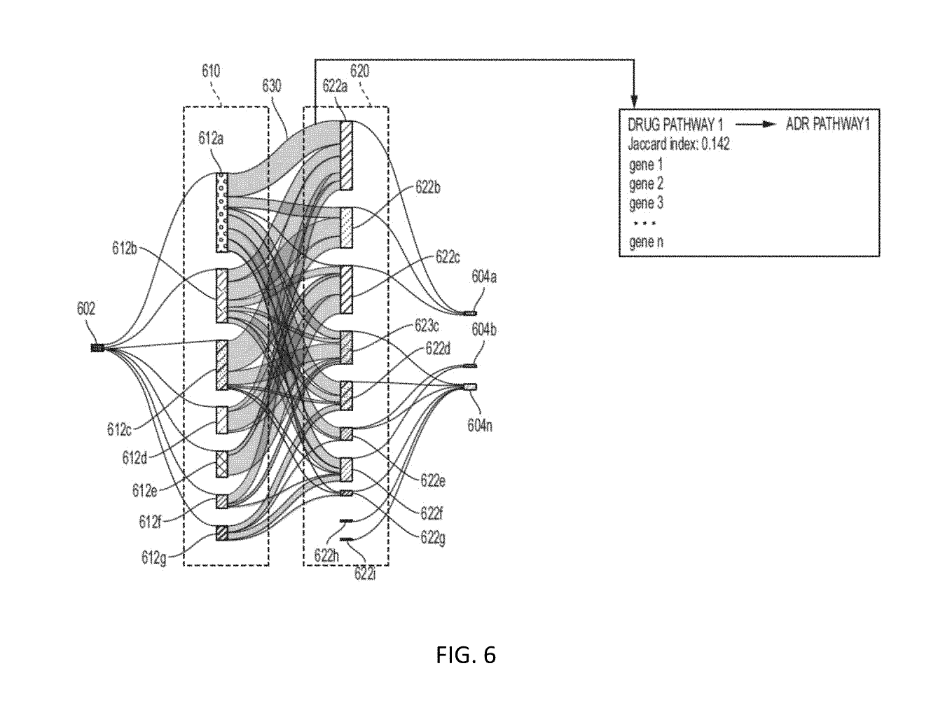

FIG. 6 depicts an exemplary system output interface according to an embodiment of the present invention. The output interface can include a drug 602, a drug pathway region 610, an ADR pathway region 620, and one or more ADRs 604a, 604b, . . . 604n. The drug pathway region 610 can include a plurality of drug pathway nodes 612a, 612b, 612c, 612d, 612e, 612f, 612g. The ADR pathway region 620 can include a plurality of ADR pathway nodes 622a, 622b, 622c, 622d, 622e, 622f, 622g, 622h, 622i. The pathway nodes represent pathways harvested from relevant databases for the indicated drug and/or ADR. As is shown, the data can be depicted in a Sankey diagram, in which the thickness of the connectors (“edges”) between pathway nodes visually represents the statistical significance of the connection between pathways. The output interface can include dynamic edges, for example such that when a user clicks or touches an edge, a graphic appears listing the indicated pathways, the genes in the pathways (gene 1, gene 2, gene 3, . . . gene n), and other relevant information such as the Jaccard index. The listing of genes can include full or partial lists of genes in the indicated pathways and can optionally highlight genes indicated in both the drug and ADR pathways through the analysis.

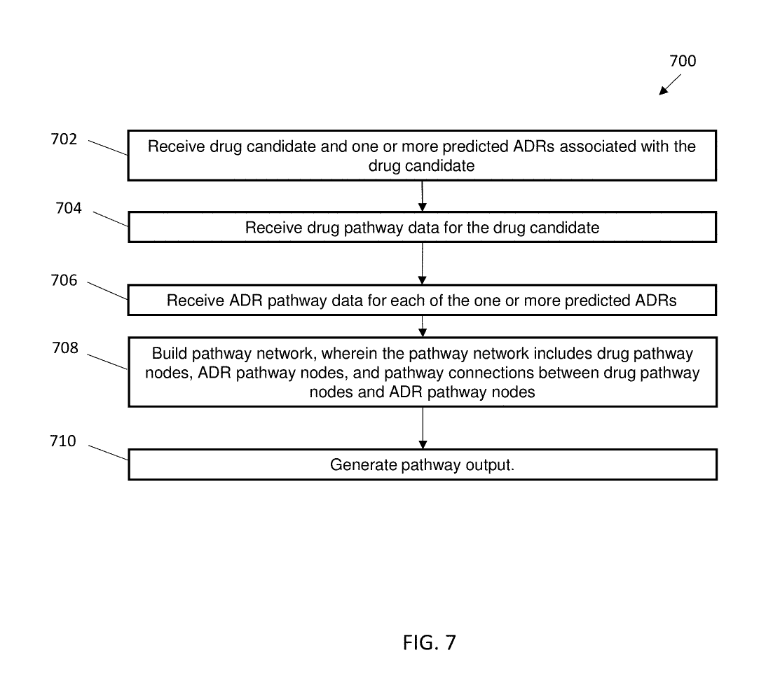

FIG. 7 depicts a flow diagram illustrating an exemplary method 700 according to an embodiment of the present invention. The method 700 can include receiving a drug candidate and one or more predicted ADRS associated with the drug candidate, as shown at block 702. For example, the drug candidate and predicted ADRs can include an output from a machine learning model. The method 700 can also include receiving drug pathway data for the drug candidate, as shown at block 704. The method 700 can also include receiving drug pathway data for the ADR, as shown at block 706. The method 700, as shown at block 708, includes building a pathway network, wherein the pathway network includes drug pathway nodes and ADR pathway nodes, and connections between drug pathways and ADR pathways. The method also includes generating a pathway output, as shown at block 710, wherein the pathway output includes predicted ADRs and drug-ADR pathway connections and related statistical rankings. In some embodiments of the invention, the mechanism of action output includes a visualized display of the pathway network and statistical rankings, for example in a Sankey diagram with one or more dynamic components.

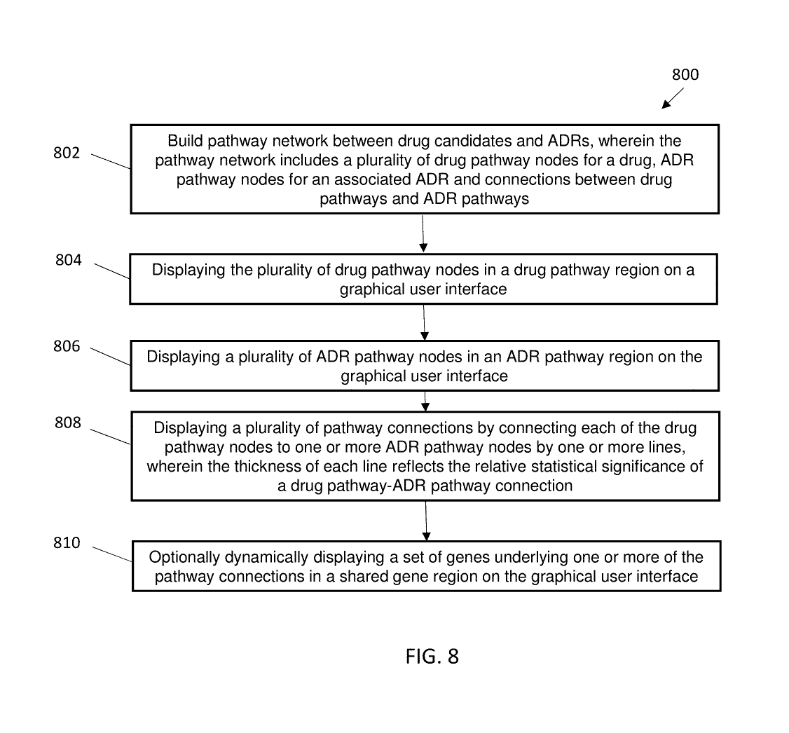

FIG. 8 depicts a flow diagram illustrating an exemplary method 800 for generating a pathway output according to another embodiment of the present invention. The method 800 includes building a pathway network between drug candidates and ADRs, wherein the pathway network includes a plurality of drug pathway nodes for a drug, ADR pathway nodes for an associated ADR, and connections between drug pathways and ADR pathways, as shown at block 802. The method also includes displaying the plurality of drug pathway nodes in a drug pathway region on a graphical user interface as shown at block 804. The method also includes displaying a plurality of ADR pathway nodes in an ADR pathway region on the graphical user interface as shown at block 806. The method also includes, as shown at block 808, displaying a plurality of pathway connections by connecting each of the drug pathway nodes to one or more ADR pathway nodes by one or more lines, wherein the thickness of each line reflects the relative statistical significance of a drug pathway-ADR pathway connection. The method also includes, as shown at block 810, optionally dynamically displaying a set of genes underlying one or more of the pathway connections in a shared gene region on the graphical user interface.

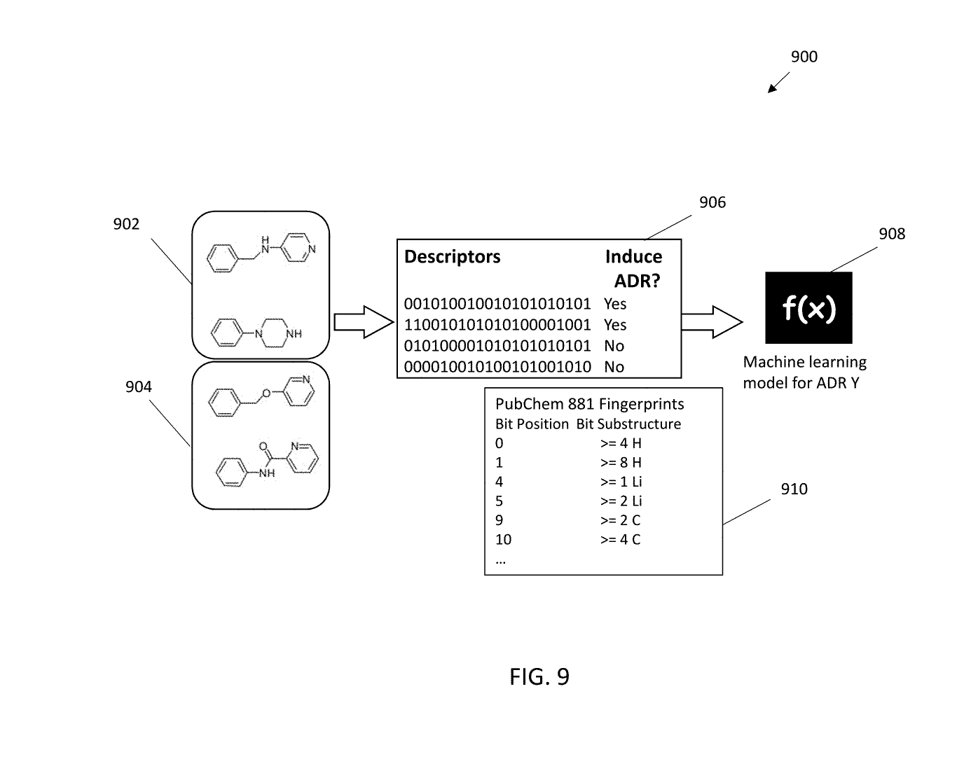

FIG. 9 depicts a schematic of machine learning-based ADR prediction according to an exemplary embodiment of the present invention. In some embodiments of the invention, methods include using a machine learning model to predict ADR(s). The machine learning model can use a positive drug set 902, including drugs known to induce a given ADR, and a negative drug set 904, including drugs unknown toward the given ADR. The machine learning model can use drug structure, such as in a SMILES format, and structure descriptors 906, for instance from fingerprints from PubChem, as seen in box 910, as input for the machine learning model 908 for the given ADR. A machine learning model can generate a set of predicted ADRs for a drug.

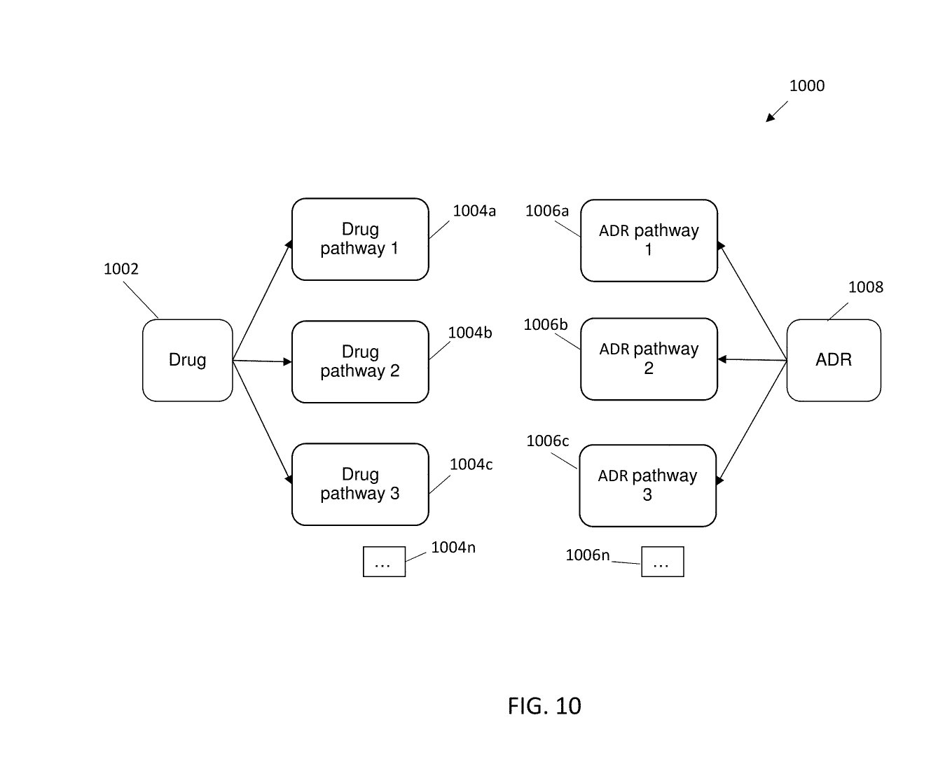

FIG. 10 depicts a schematic of pathway collection 1000 according to an exemplary embodiment of the present invention. A drug 1002 and ADR 1008, for example derived from machine learning model, can be received as input. As is shown, for a selected drug 1002, a plurality of drug pathways 1004a, 1004b, 1004c, . . . 1004n can be harvested from one or more known pathway databases, such as Drug-Path. For the given ADR 1008, a plurality of ADR pathways 1006a, 1006b, 1006c, . . . 1006n can be harvested from known pathway data sources, for instance by curating pathway information from the literature (e.g., KEGG, Reactome, etc.).

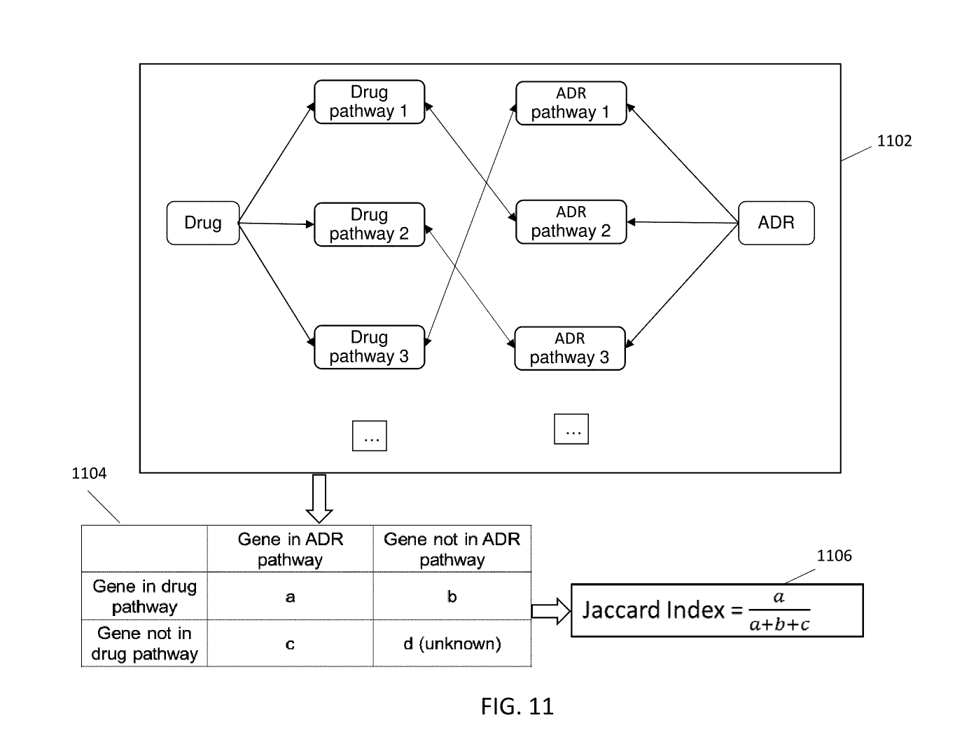

FIG. 11 depicts a schematic of pathway connection and statistical analysis according to an exemplary embodiment of the present invention. For example, starting with the drug pathway and ADR pathway data collected as demonstrated in FIG. 10, drug pathways and ADR pathways can be analyzed for associations by shared genes. Connections between drug pathways and ADR pathways can be formed as a result of such analysis to form a pathway network 1102. Genes in and not in ADR pathways can be mapped against genes in and not in drug pathways, as is illustrated at 1104 in FIG. 11, and a Jaccard Index 1106 can determined by known methods and output.

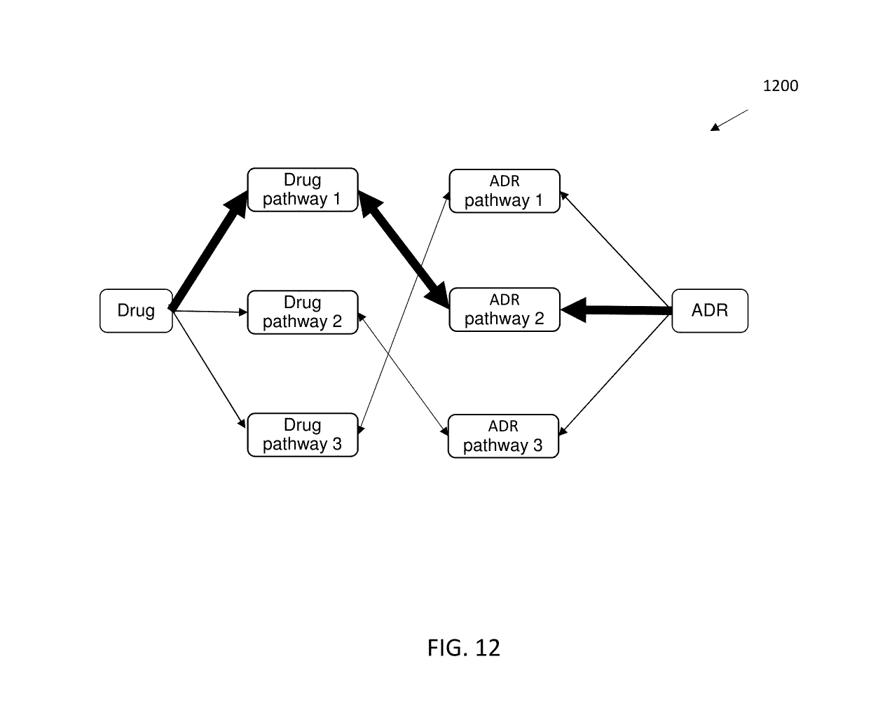

FIG. 12 depicts an exemplary visualization 1200 and hypothesis generation according to embodiments of the present invention. Pathway connections can be statistically ranked, for example by Jaccard Indexes, and top connections can be emphasized, for example by emphasizing pathway connections through line thickness. Drug-ADR pathway connections function as mechanism of action hypotheses by providing a rationale and/or reason as to how a drug induces an ADR. The gene shared by the drug and ADR pathways can be starting points for wet-lab hypothesis testing.

For example, in an early drug development stage, pharmaceutical companies can use systems and methods according to embodiments of the present invention to predict potential ADRs for drug candidates and identify the underlying mechanisms of actions. The companies can thereafter modify the drug candidates to avoid the ADRs and improve safety.

By way of another example, in the post-market stage of pharmaceutical development, pharmaceutical companies can use systems and methods according to embodiments of the invention to identify the mechanisms of actions for ADRs associated with their pharmaceutical products. By studying the pathways and genes identified in the shared pathways, it is possible to find genetic biomarkers susceptible to certain ADRs. Thus, medical providers and pharmaceutical drug providers can advise patients having the identified biomarkers to adjust doses or prescriptions to avoid the ADRs (referred to as “precision medicine”).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments of the invention, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form described. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The flow diagrams depicted herein are just one example. There can be many variations to this diagram or the steps (or operations) described therein without departing from the spirit of embodiments of the invention. For instance, the steps can be performed in a differing order or steps can be added, deleted or modified. All of these variations are considered a part of the claimed invention.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

### EXAMPLES

### Example 1

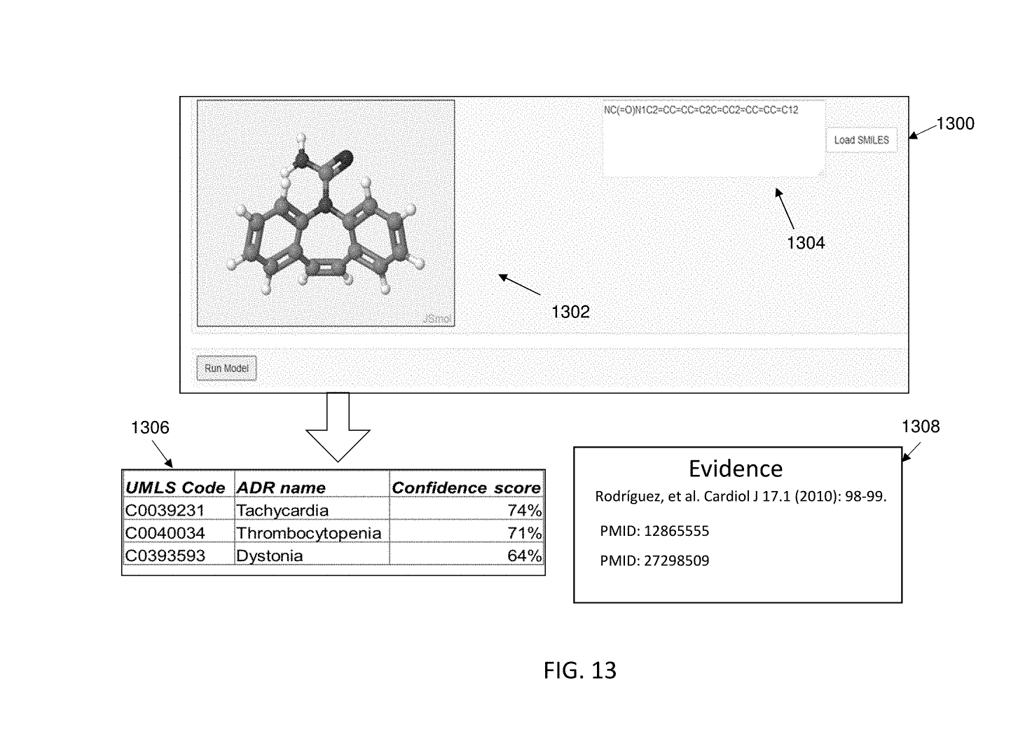

A mechanism of action hypothesis was derived for carbamazepine according to embodiments of the present invention. As is illustrated in FIG. 13, a drug structure 1302 and SMILES data 1304 were provided to a user interface 1300 of a predictive machine learning model. The model generated a plurality of predicted ADRs and related confidence scores at an output 1306. Evidence for the ADRs 1308 was also output.

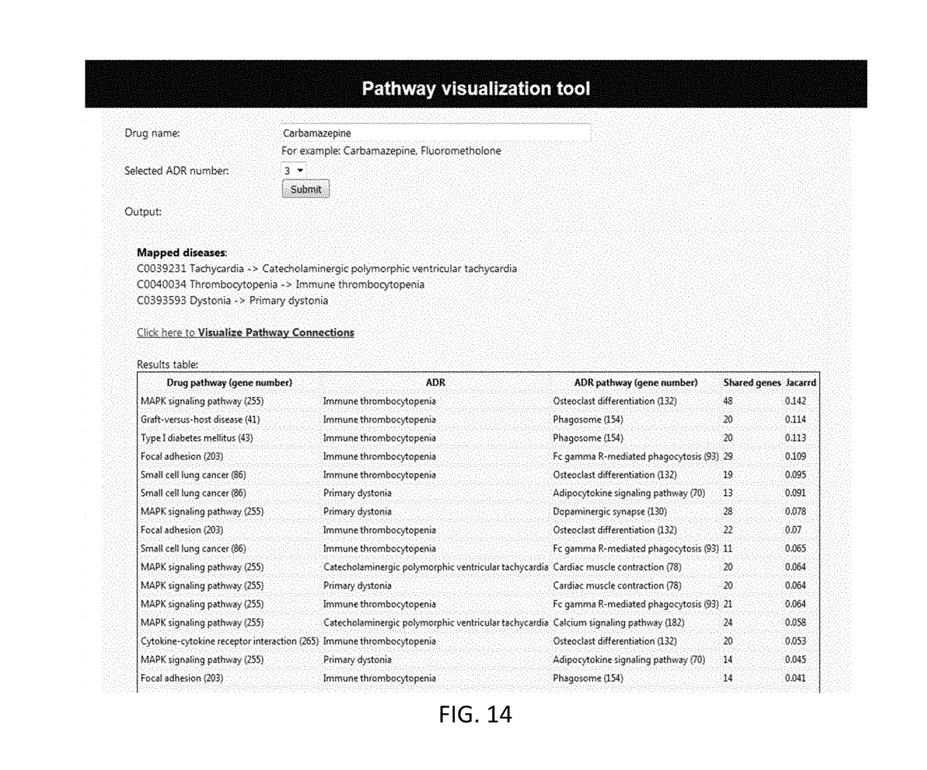

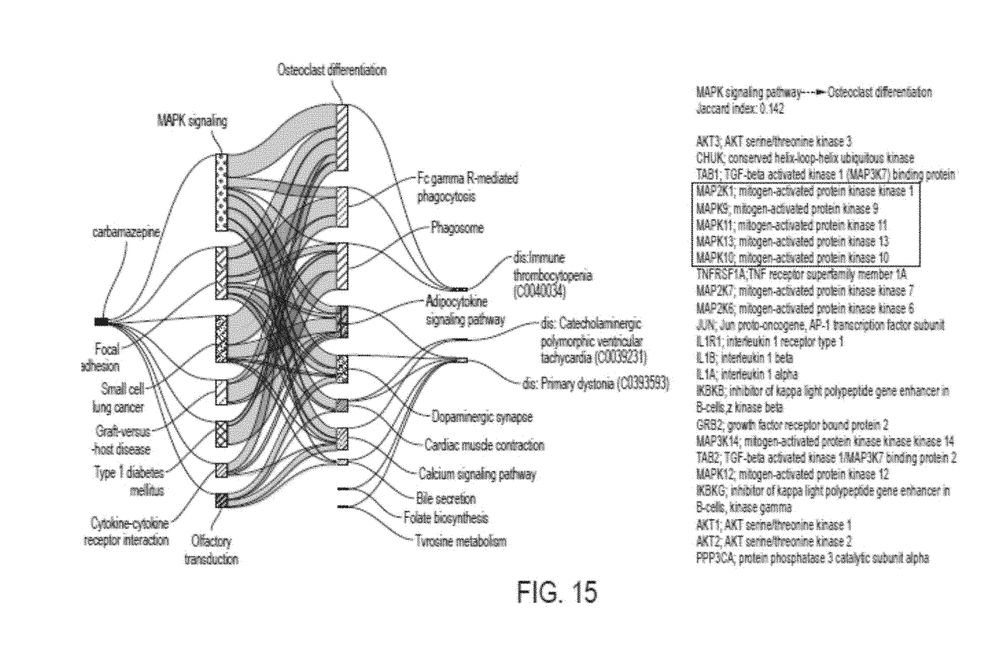

A pathway association analysis was performed on the predicted ADRs and drug, as is depicted in FIG. 14. The exemplary pathway visualization tool generated a listing of drug pathways and ADR pathways in a graphical user interface, along with a number of shared genes and Jaccard index. The pathways were ranked according to Jaccard Index, as is shown. For example, the leading pathway connection for carbamazepine for the immune thrombocytopenia ADR was through the MAPK signaling pathway and Osteoclast differentiation pathway, including 48 shared genes and having a Jaccard Index of 0.142. The exemplary graphical user interface includes a dynamic component, in which a user was provided an option to “click here” to visualize pathway connections. FIG. 15 depicts the visualized Drug-ADR pathway analysis output for carbamazepine. Drug pathways and ADR pathways are visualized using a Sankey diagram and genes shared by the MAPK signaling pathway and Osteoclast differentiation pathway were revealed by clicking on the edge connecting these ADRs in the Sankey diagram. The gene listing appears on the right of FIG. 14. Further analysis of the listing was performed and MAP2K1, MAPK9, MAPK 11, MAPK13, and MAPK10 (the fourth through eighth genes identified in the listing of genes) were reported in the literature to be associated with the connection.

### Example 2

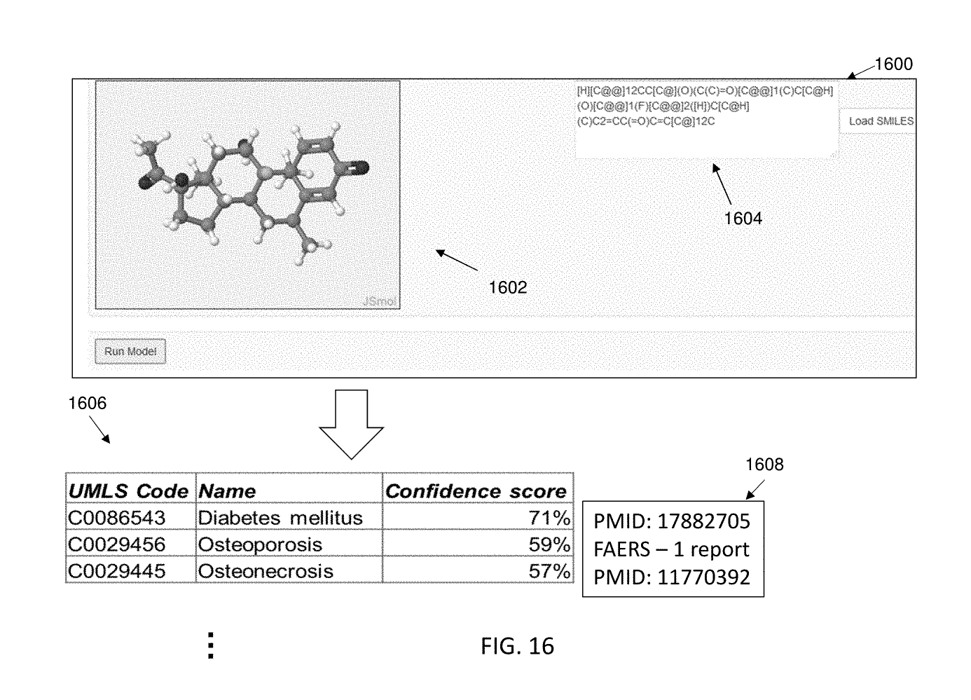

A mechanism of action hypothesis was derived for fluorometholone according to embodiments of the present invention. As is illustrated in FIG. 16, a drug structure 1602 and SMILES data 1604 were provided to a user interface 1600 of a predictive machine learning model. The model generated a plurality of predicted ADRs and related confidence scores at an output 1606. Evidence for the ADRs 1608 was also output.

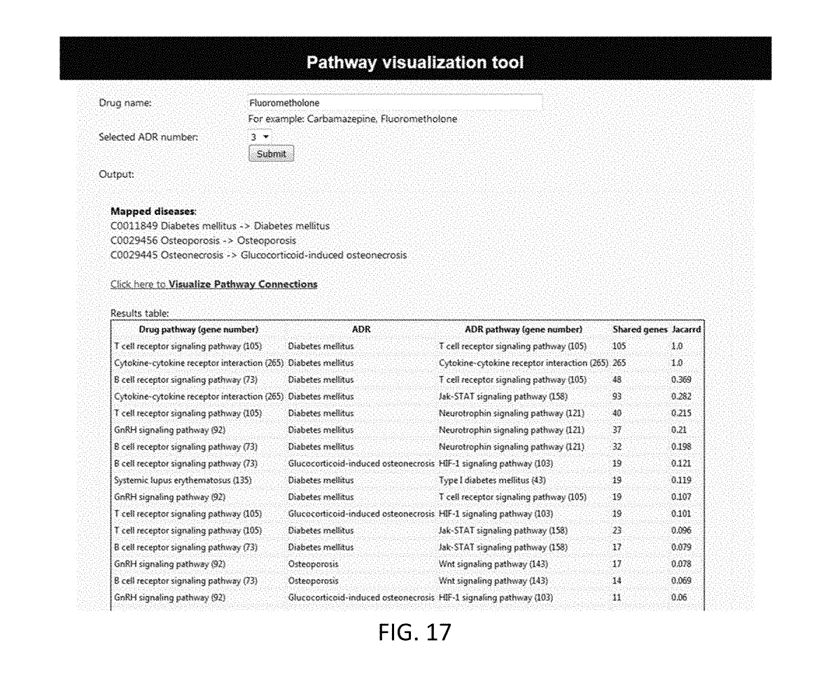

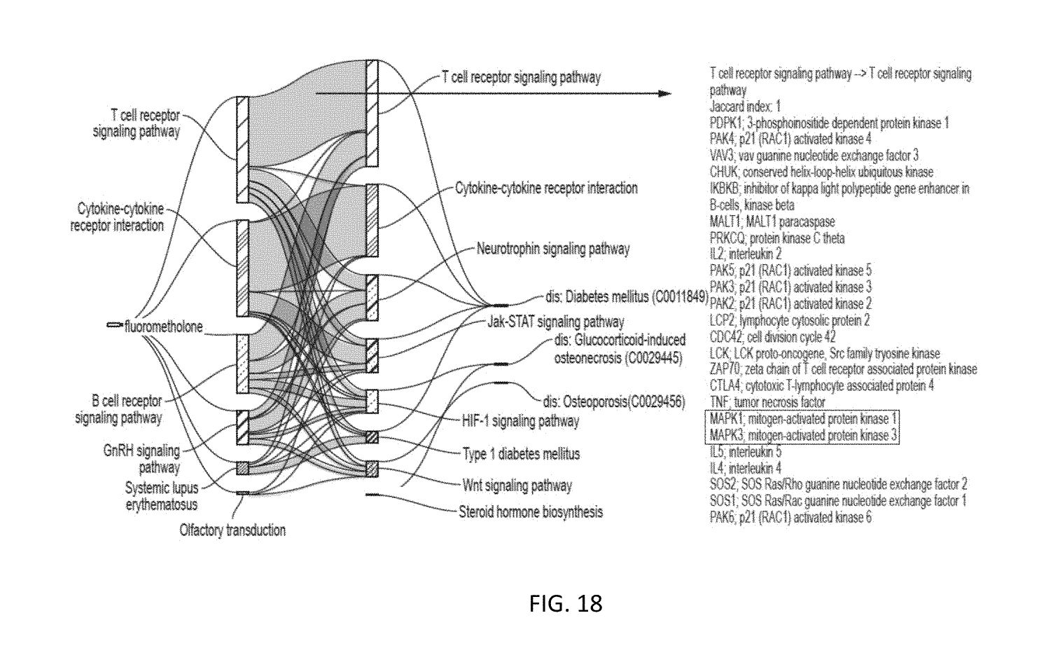

A pathway association analysis was performed on the predicted ADRs and drug, as is depicted in FIG. 17. The exemplary pathway visualization tool generated a listing of drug pathways and ADR pathways in a graphical user interface, along with a number of shared genes and Jaccard index. The pathways were ranked according to Jaccard Index, as is shown. For example, the leading pathway connection for fluorometholone for the diabetes mellitus ADR was through the T cell receptor signaling pathway, including 105 shared genes and having a Jaccard Index of 1.0. The exemplary graphical user interface includes a dynamic component, in which a user was provided an option to “click here” to visualize pathway connections. FIG. 18 depicts the visualized Drug-ADR pathway analysis output for flurometholone. Drug pathways and ADR pathways are visualized using a Sankey diagram. In this example, both the drug and ADR were determined to have a common pathway, the T cell receptor signaling pathway. The gene listing, displayed after clicking the top edge of the Sankey diagram, appears on the right of FIG. 18.

What is claimed is:

1. A computer-implemented method for generating a mechanism of action hypothesis for an adverse drug reaction, the method comprising:
receiving, by a processor, drug candidate data that identifies a drug candidate along with and a plurality of predicted adverse drug reactions associated with the drug candidate data;
receiving, by the processor, drug pathway data for the drug candidate;
receiving, by the processor, adverse drug reaction pathway data for each of the plurality of predicted adverse drug reactions;
building, by the processor, a pathway network, wherein the pathway network comprises a plurality of drug pathway nodes, a plurality of adverse drug reaction pathway nodes, and a plurality of pathway connections; and
generating a pathway output.

2. The computer-implemented method of claim 1, wherein the pathway output comprises a visualized output for the pathway connections.

3. The computer-implemented method of claim 2, wherein the visualized output visually depicts the statistical significance of each of the pathway connections.

4. The computer-implemented method of claim 1, further comprising a dynamic pathway output comprising a list of genes for one of the connections between the drug pathway nodes and the adverse drug reaction nodes.

5. The computer-implemented method of claim 1, wherein building the pathway network comprises identifying pathway connections between drug pathways for the drug and adverse drug reaction pathways for the adverse drug reaction and statistically analyzing the pathway connections.

6. The computer-implemented method of claim 5, wherein statistically analyzing the pathway connections comprises applying a Jaccard Index to the pathway connections.

7. The computer-implemented method of claim 1, further comprising applying a machine learning model to the drug candidate to generate the plurality of predicted adverse drug reactions.

8. A computer-implemented method for displaying a mechanism of action hypothesis for an adverse drug reaction, the method comprising:
building a pathway network between drug candidates and adverse drug reactions (ADRs), wherein the pathway network comprises a plurality of drug pathway nodes for a drug, a plurality of ADR nodes for an associated ADR, and connections between the drug pathways and ADR pathways;
displaying the plurality of drug pathway nodes in a drug pathway region on a graphical user interface;
displaying a plurality of ADR pathway nodes in an ADR pathway region on the graphical user interface; and
displaying a plurality of pathway connections by connecting one or more of the drug pathway nodes to one or more of the ADR pathway nodes by one or more lines, wherein the relative thickness of each of the lines reflects the statistical significance of a drug pathway-ADR pathway connection.

9. The computer-implemented method of claim 8, further comprising dynamically displaying a set of genes underlying one or more of the pathway connections in a shared gene region on the graphical user interface.

10. The computer-implemented method of claim 8, wherein displaying a plurality of pathway connections comprises displaying a Sankey diagram.
